(19) United States
(12) Patent Application Publication
(10) Pub. No.: US 2010/0240968 A1
(43) Pub. Date: Sep. 23, 2010

(54) AUTOMATIC DATE AND TIME ACQUISITION IN BIOMETRIC MONITORS

(76) Inventor: Thomas Zeindler, Fort Myers, FL (US)

Correspondence Address: J. Rodman Steele;Novak Druce & Quigg LLP, 525 Okeechobee Blvd, Suite 1500, West Palm Beach, FL, 33401, US

(21) Appl. No.: 12/718,491

(22) Filed: Mar. 5, 2010

Related U.S. Application Data
(60) Provisional application No. 61/158,053, filed on Mar. 6, 2009.

(51) Int. Cl. A61B 5/00

(52) U.S. Cl. 600/301

(57) ABSTRACT

A biometric monitoring system configured for taking biometric measurements of a patient is described herein. The system can include a biometric measurement unit configured to take one or more biometric measurements and a processor coupled to the biometric measurement unit. The processor can be operable to receive the biometric measurements from the biometric measurement unit. The system can also include a clock that can set timing in the biometric monitoring system and a receiver that can receive atomic clock time information. The atomic clock time information can be used to synchronize the clock.

### CROSS REFERENCE TO RELATED APPLICATIONS

This application claims the benefit of U.S. Provisional Application No. 61/158,053, entitled “Automatic Date and Time Acquisition in Automated Blood Pressure Monitors,” filed Mar. 6, 2009, the entirety of which is incorporated herein by reference.

### FIELD OF TECHNOLOGY

The subject matter herein is related to the field of automated biometric monitoring, and more particularly to automated biometric monitors that utilize date and time programming of biometric data.

### BACKGROUND

The monitoring of blood pressure or other biometric parameters has become an indispensible tool in many aspects of medicine. Monitoring a patient at home using known devices like blood pressure monitors and transferring the information to a caregiver is a fast growing emerging part of telemedicine and medical services.

Accurately keeping track of blood pressure readings over time is an integral part of treating hypertension. With recent advancements in telemedicine, patients at home can send blood pressure readings/data to a physician without having to physically visit the physician by utilizing blood pressure monitoring devices capable of connecting to a computer and sending the blood pressure data over a communications network. For a patient that is required to regularly monitor and submit blood pressure data, it is important to keep track of the data by time of day. Without a date and time, merely sending a physician a list of blood pressure values cannot allow the physician to analyze the progress of the patient's blood pressure over time.

Date and time information is the basis for analyzing cumulative blood pressure readings over time. Therefore, ensuring that the time and date information is accurately programmed in a monitoring device is pertinent to treating hypertension. Currently, there are available monitoring devices that allow a user to manually program the date and time feature, however, most often, elderly patients find it challenging and often times difficult to program a feature for a device. Even if initially programmed, current monitoring devices with time and date features often require re-programming due to power outages or when replacing the batteries of the monitoring device. Additionally, when travelling, it is a burden to re-program the monitoring device to accurately record the blood pressure reading taken by the patient at the time of reading.

### SUMMARY

A biometric monitoring system configured for taking biometric measurements of a patient is described herein. In one arrangement, the system can include a biometric measurement unit that can be configured to take one or more biometric measurements and a processor that can be coupled to the biometric measurement unit. In another arrangement, the processor can be operable to receive the biometric measurements from the biometric measurement unit. The system can also include a clock that can set timing in the biometric monitoring system and a receiver that can receive atomic clock time information. The atomic clock time information can be used to synchronize the clock. The biometric system can also include a storage unit that can be coupled to the processor, which can store the biometric measurements taken by the biometric measurement unit.

In another arrangement, the processor can be operable to determine when a previous atomic clock time information was received, and the receiver can be operable to receive atomic clock time information if the processor determines that the previous atomic clock time information was received outside a predetermined time period. An example of a predetermined time period can be seven days.

The system can also take biometric measurements in accordance with a plurality of time intervals. Examples of time intervals can include a morning session and an evening session. Non-limiting examples of biometric measurements that can be taken by the system include a blood pressure reading, a heart rate, a blood sugar reading, a cholesterol reading or a body temperature reading. The system can also include a transceiver that transmits the biometric measurements taken by the biometric measurement unit to a remote location at a defined interval.

Another biometric monitoring system is described herein. This system can include a receiver that can be configured to receive atomic clock time information and a processor that can be coupled to the receiver. The processor can also be operable to time stamp a biometric measurement based on the atomic clock time information. The system can also include a biometric measurement unit that can be configured to take one or more biometric measurements and forward the biometric measurements to the processor. The system can also include a storage unit that can be coupled to the processor, which can store the biometric measurements for a predetermined time period. The system can also include a transceiver configured to at least transmit the stamped biometric measurement to a remote location at a defined interval.

A method of automatically updating time information in a biometric monitoring system is also described herein. In one arrangement, the method can include the steps of receiving atomic clock time information and stamping a biometric measurement based on the atomic clock time information.

In another arrangement, receiving the atomic clock time information can include identifying when a previous atomic clock time information was received and receiving the atomic clock time information if the previous atomic clock time information was received outside a predetermined time period. As noted earlier, an example of the predetermined time period is seven days.

In another arrangement, the method can include taking the biometric measurement. Taking the biometric measurement can also include taking a plurality of biometric measurements at multiple intervals and comparing the biometric measurements taken at different intervals with one another. Examples of different intervals can include a morning session and an evening session. The method can also include the steps of storing the biometric measurements and transmitting the biometric measurements to a remote location at a defined interval. Examples of biometric measurements that can be taken include a blood pressure reading, a heart rate, a blood sugar reading, a cholesterol reading or a body temperature reading.

Additional aspects will be set forth in part in the description which follows, and in part will be obvious from the description, or may be learned by practice of the subject matter herein. These aspects will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute part of this specification, illustrate embodiments and together with the description, serve to explain the principles of the subject matter herein. The embodiments illustrated herein are presently preferred, it being understood, however, that the subject matter is not limited to the precise arrangements and instrumentalities shown, wherein:

FIG. 1 is an illustration of an example of an automatic biometric monitoring system for automatically updating time information;

FIG. 2 is a schematic illustration of the biometric monitoring system of FIG. 1; and

FIG. 3 is a flow chart illustrating an exemplary process for automatically updating time information in an automatic biometric monitoring system.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein.

Several definitions that apply throughout this document will now be presented. The word “coupled” is defined as connected or integrated with, whether directly or indirectly through intervening components, and is not necessarily limited to physical connections. The term “biometric measurement” is defined as a measurement of a physical characteristic of an animal or human. A “biometric measurement unit” is defined as a component or a group of components capable of taking biometric measurements. A “processor” is defined as a component or a group of components capable of executing tasks or instructions that are in accordance with any of the processes described herein. The word “clock” is defined as an instrument for measuring or indicating time. The term “storage unit” means a component or group of components capable of receiving and at least temporarily holding information. The phrase “morning session” means the first part or period of a day. The phrase “evening session” means the last part or second period of a day. A “transceiver” is defined as a component or group of components capable of transmitting and/or receiving information over a wireless or hard-wired connection. A “remote location” is defined as a location that is physically discrete from the location where biometric measurements take place.

As noted earlier, accurately keeping track of blood pressure or other biometric parameters over time is an integral part of treating patients. However, manually programming the date and time feature in current monitoring devices, which is required by power outages, battery replacement, or travelling, is subject to operator error and/or re-programming. Therefore, it is difficult to ensure that the time and date information is accurately programmed in a monitoring device.

To overcome these difficulties, a biometric monitoring system configured for taking biometric measurements of a patient is presented herein. In one arrangement, the system can include a biometric measurement unit that can be configured to take one or more biometric measurements and a processor that can be coupled to the biometric measurement unit. The processor can be operable to receive the biometric measurements from the biometric measurement unit. The system can also include a clock that can set timing in the biometric monitoring system and a receiver that can receive atomic clock time information. The atomic clock time information can be used to synchronize the clock. In view of the accuracy of atomic clock time information, automatically synchronizing a biometric monitoring device with such information is efficient and uncomplicated for the user. As a result, the biometric monitoring system presented herein provides an effective way to accurately keep track of biometric measurements over time.

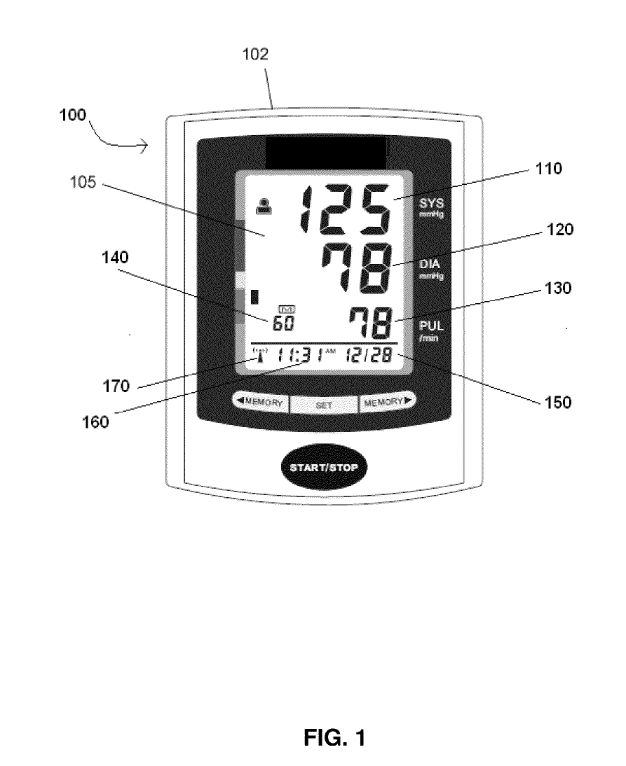

FIG. 1 is an illustration of a biometric monitoring system 100 configured for taking biometric measurements of a patient. As an example, the biometric monitoring system 100 can include a biometric measurement unit 102 that is configured to take one or more biometric measurements. In one particular example, but without limitation, the biometric measurement unit 102 can be a blood pressure monitor, although it is understood that the unit 102 can be any other suitable device for monitoring a wide variety of biometric parameters. Although not pictured, the system 100 can include various components for taking blood pressure, as is known in the art, such as a cuff. In this case, the system 100 can include a display 105 and can render blood pressure readings of both systolic pressure 110 and diastolic pressure 120, as well as an average pulse 130 at the time of reading. Another feature rendered can be a memory status 140 shown in the display 105. In one arrangement, the system 100 can be automatically updated by receiving atomic clock time information. Notably, an atomic clock time icon 170 can be displayed to indicate that the time 160 and date 150 information is based on an up-to-date atomic clock time signal received by the system 100.

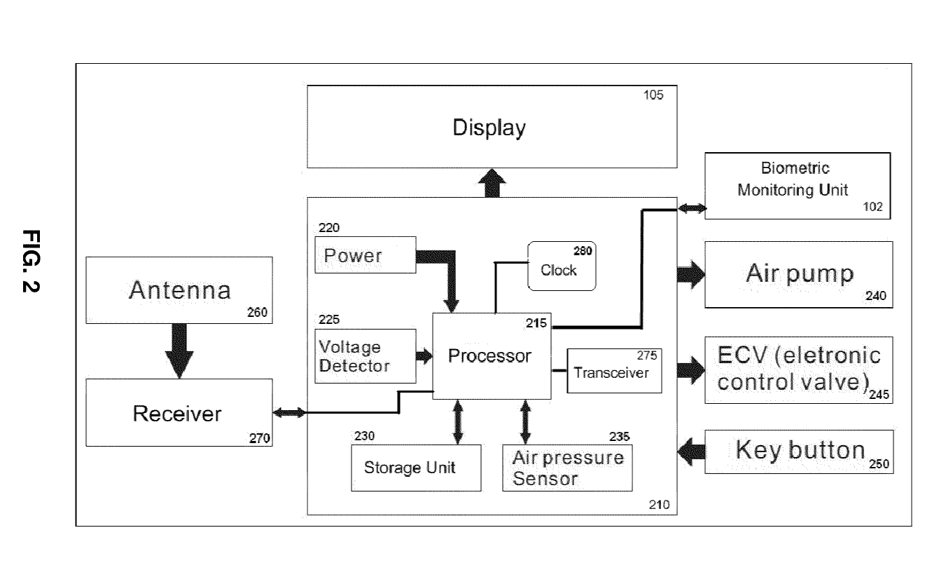

FIG. 2 shows an exemplary block diagram of some of the components of the biometric monitoring system 100 of FIG. 1. In one implementation, the system 100 can include a main electronic board 210, a processor 215, an air pressure sensor 235, a storage unit 230, a voltage detector 225 and a power source 220. The system 100 can also include an air pump 240, configured to inflate a pneumatic system, and an electronic-controlled valve (ECV) 245 configured to deflate the pneumatic system.

The air pressure sensor 235 can measure the air pressure in a cuff (not shown) that is used by an operator during a blood pressure reading process. In addition, the storage unit 230 can receive biometric measurement data and can store this data until it is retrieved or deleted based on the age of the data. For example, the storage unit 230 can hold up to sixty blood pressure readings before deleting the oldest stored readings as new data comes in. The power source 220 can provide appropriate power to the components of the system 100. Key buttons 250, such as start/stop, allow a user to interact with the display 105 and to input commands to the system 100. The system 100 can also include a transceiver 275 that can send signals to (or receive from) a remote location. These signals can include information generated by the system 100. The transceiver 275 can also send (or receive) wireless signals or can be coupled to a suitable interface for a hard-wired connection. As noted earlier, the system 100 can include a biometric monitoring unit 102. The unit 102 can take biometric measurements of a patient and can forward them to the processor 215. The biometric measurements can be automatically initiated by the system 100 based on a predetermined interval, or an operator can simply activate the measurement process on his/her own choosing. Examples of these biometric measurements will be presented below.

The system 100 can also include a clock 280 that sets timing for the components of the system 100. The clock 280 can be coupled to the processor 215 and can receive updates at suitable times from the processor 215. Notably, the clock 280 can be contained within the processor 215, or alternatively, the clock 280 can be a discrete component separate from and coupled to the processor 215.

The system 100 can include an antenna 260 coupled to a receiver 270, and the receiver 270 can be coupled to the processor 215. Through the antenna 260, the receiver 270 can receive information from, for example, over-the-air updates and can process and provide the information to the processor 215. As an example, the information can be atomic clock time information, although it is understood that the information can include other suitable forms of data. At a suitable time, the processor 215 can synchronize the clock 280 with the atomic clock time information, which can enable the system 100 to generate temporally accurate biometric measurements.

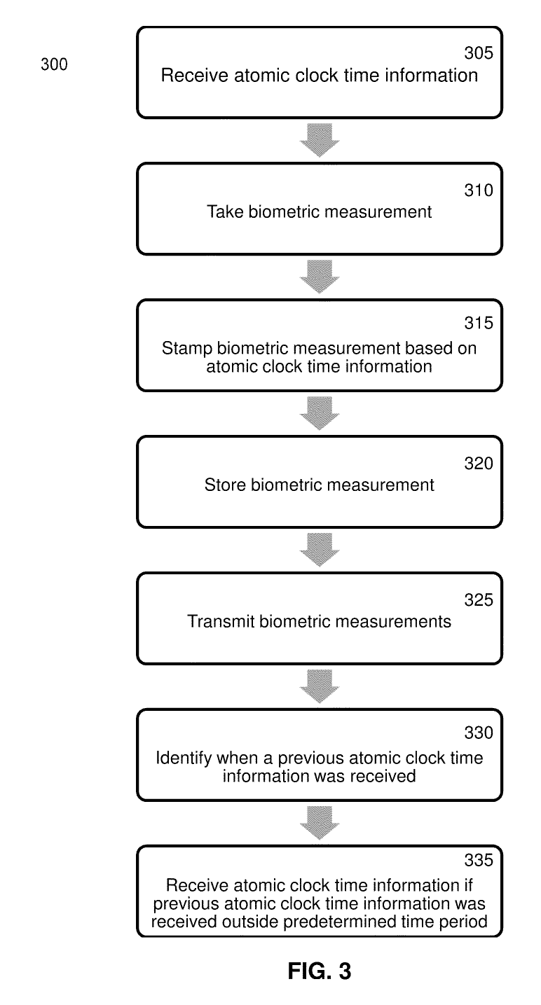

Referring to FIG. 3, a method 300 of automatically updating time information is shown. To describe this method, reference may be made to the components of FIGS. 1 and 2, although the method can be applicable to or practiced with other structures. Moreover, the steps of the method 300 are not limited to the order in which they are presented here, and the method 300 may include a greater (or even fewer) number of steps than what is shown.

At step 305, atomic clock time information can be received, and at step 310, one or more biometric measurements can be taken. At step 315, the biometric measurement can be stamped based on the atomic clock time information. In addition, the biometric measurement can be stored, as shown in step 320. At step 325, these biometric measurements can be transmitted to, for example, a remote location.

As noted earlier, the clock 280 is useful for setting timing in the biometric monitoring system. To ensure its accuracy, the clock 280 can be updated with external timing information, such as atomic clock time information. To that effect, the antenna 260 can receive signals that contain such information and can forward these captured signals to the receiver 270. As an example, the antenna 260 of the biometric monitoring system 100 can receive radio signals that keep the system 100 in synchronization with the atomic clock of the U.S. National Institute of Standards & Technology (NIST), Time & Frequency Division, in Boulder, Colo.

The WWVB radio transmitter, located in the Fort Collins, Colo., continuously broadcasts a time signal based on this atomic clock throughout the United States at 60 kHz to take advantage of stable long wave radio paths found in that frequency range. In one implementation, the antenna 260 of the biometric monitoring system 100 can receive these relatively low frequency signals and can provide them to the receiver 270. The receiver 270 can then process these incoming signals and can provide the corresponding atomic clock time information to the processor 215. The processor 215 can then use the atomic clock time information to synchronize the clock 280. Thus, the timing of the system 100 can be precisely maintained, which can permit temporally accurate biometric measurements to be recorded. This principle can apply even in view of mandated time changes, like daylight savings programs or leap years.

The atomic clock time information can include various types of information. In addition to time and date, the atomic clock time information can include information relating to weather events, emergency messages, health reports, geophysical alerts and any other suitable broadcasts. This information can be processed to be displayed on the display 105 or can be played through a speaker (not shown) of the biometric monitoring system 100. The atomic clock time information can also include voice broadcasts, whether human or artificially generated signals. The term “atomic clock time information” is defined as information that at least includes timing that is based on an atomic clock.

It must be pointed out that the biometric monitoring system 100 is not limited to being updated with atomic clock time information. For example, other timing synchronization signals can be received from other suitable systems, such as GPS signals or signals from a base station of a communications network. Moreover, the system 100 can receive information relating to updates of other aspects of the system, like software updates for the processor 215 or the biometric monitoring unit 102. These updates can be received by the transceiver 275, for example. Additionally, the system can receive signals from other organizations that provide atomic clock time information, such as WWV, also based in Colorado, WWVH based in Hawaii, DCF77 out of Germany, HBG in Switzerland, JJY from Japan, MSF based in the United Kingdom and TDF in France. It must also be pointed out that the antenna 260 can be configured to resonate at a single frequency or multiple frequencies and certainly is not limited to 60 kHz signals, as higher or even lower frequencies can be captured based on its design.

An operator may wish to have a biometric measurement recorded. As such, the operator may engage the biometric measurement unit 102 of the biometric monitoring system 100. In one particular example, the biometric monitoring system 100 can be a blood pressure monitor, and the biometric measurement unit 102 can take blood pressure readings. In this case, the operator can attach a cuff (not shown) to his/her arm and can initiate a measurement by activating the key button 250. This step can cause the processor 215 to activate the air pump 240, and the air pressure sensor 235 can provide feedback as to the air pressure generated by the air pump 240. Once the air pressure reaches an acceptable level, the processor 215 can signal the air pump 240 to deactivate. The biometric measurement unit 102 can then take blood pressure readings and can forward them to the processor 215. The processor 215 can stamp the blood pressure readings with date and/or time information from the clock 280 and can signal the ECV245 to release the air pressure. As discussed earlier, the date and/or time information of the clock 280 is based on atomic clock time information. The blood pressure readings can then be sent to the storage unit 230, which stores the blood pressure readings taken by the blood pressure monitor.

A plurality of biometric measurements can be taken by the biometric monitoring system 100 at multiple intervals and stored in the storage unit 230. In the particular example of a blood pressure monitor, the operator may wish to take blood pressure readings at least twice a day, once during a morning session and once during an evening session. For example, a morning session reading can be taken shortly after the operator wakes up, and an evening session reading can be taken shortly before the operator goes to bed. However, it is understood that the morning and evening sessions may include any suitable time of day. The morning and evening sessions may also be separated by a minimum predetermined time period, if desired.

The plurality of biometric measurements taken by the biometric monitoring system 100 can be compared with one another. For example, an average may be calculated for some or all of the biometric measurements stored in the storage unit 230 of the system 100. In another example, biometric measurements taken at different intervals can be compared with one another. In the particular example of a blood pressure monitor, the operator may wish to compare his/her morning and evening blood pressure readings. Therefore, the system 100 can calculate the average of all blood pressure readings taken during morning sessions and stored in the storage unit 230, calculate the average of all blood pressure readings taken during evening sessions and stored in the storage unit 230, and compare the two averages. As an example, the calculations described above may be performed by the processor 215 or some other component in the system 100 and can also be stored in the storage unit 230. Alternatively, the raw data from the system 100 can be forwarded to an external unit, such as a computer (not shown), which can also perform the calculations.

To transmit the biometric measurements taken by the system 100, the transceiver 275 of the system 100 can transfer the biometric measurements and any other relevant data stored in the storage unit 230 to a remote location at a defined interval. The biometric measurements can be transmitted over a wireless or hard-wired connection. The remote location can include an external computer, a physician's office, a hospital, other medical facilities or any other desired location. The defined interval can be based on time, the number of measurements stored in the storage unit 215, an operator's initiation or any other suitable event. Thus, a caretaker or some other entity or organization responsible for caring for a patient can receive updated and accurately time-stamped biometric measurements from the system 100, thereby improving patient care.

In one implementation, the defined interval can be based on a predetermined time period, such as one month. In this case, the transceiver 275 can transmit the relevant information to the remote location once a month. In another example, if the defined interval is based on a number of biometric measurements, the transceiver 275 can forward the relevant information following the completion of 60 biometric measurements. As another example, an operator may cause the transceiver 275 to forward relevant information if the operator believes that a biometric measurement that was recently taken is outside a safe range or presents an otherwise abnormal reading. Other suitable values can be employed when determining a defined interval. For example, the defined interval can be a different time period or a different number of measurements or can even be based on some other predetermined event. Additionally, the defined interval is not limited to any single parameter.

As explained earlier, the biometric measurements of the biometric monitoring system 100 are not limited to blood pressure readings. Other types of biometric measurements can include a heart rate, a blood sugar reading, a cholesterol reading, a body temperature reading or any other suitable biometric measurement that may require monitoring. Additionally, the biometric measurements are not limited to a single type of biometric measurement. For example, in one implementation, the system 100 can include a blood pressure monitor configured for irregular heartbeat detection and the display 105 can present a hypertension indicator (not shown) or an irregular heartbeat alert (not shown).

Referring back to FIG. 3, at step 330, the system 100 can identify when a previous atomic clock time information was received, and at step 335, the system 100 can receive atomic clock time information if the previous atomic clock time information was received outside a predetermined time period.

In one implementation, the processor 215 of the biometric monitoring system 100 can identify when a previous atomic clock time information was received. If the previous atomic clock time information was received outside a predetermined time period, then the system 100 can receive new atomic clock time information as described earlier. That is, the processor 215 can signal the receiver 270 to receive the signals that contain the atomic clock time information and to process these signals for updating the clock 280. Examples of a predetermined time period include 24 hours, seven days or one week, though it is understood that the predetermined time period can be any suitable period of time.

Other aspects and features can include capability of the biometric monitoring system 100 of being linked to a computer for downloading cumulative data utilizing a biometric monitoring software/computer program product. The program product can facilitate the analysis of the biometric measurements and can sort the data based on the time and date attached to each entry. The program product can transform the data into different customized charts/graphs that can be sent to a physician's office or other medical facility for further analysis.

Embodiments of the system 100 or method 300 can take the form of an entirely hardware embodiment, an entirely software embodiment or an embodiment containing both hardware and software elements. In one embodiment, the method 300 is implemented in software, which includes but is not limited to firmware, resident software, microcode, and the like. Furthermore, the method 300 can take the form of a computer program product accessible from a computer-usable or computer-readable medium providing program code for use by or in connection with a computer or any instruction execution system.

For the purposes of this description, a computer-usable or computer readable medium can be any apparatus that can contain, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk or an optical disk. Current examples of optical disks include compact disk-read only memory (CD-ROM), compact disk-read/write (CD-R/W) or DVD.

A data processing system suitable for storing and/or executing program code and for use with the subject matter described herein can include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. Input/output or I/O devices (including but not limited to keyboards, displays, pointing devices, etc.) can be coupled to the system either directly or through intervening I/O controllers. Network adapters can also be coupled to the system to enable the data processing system to become coupled to other data processing systems or remote printers or storage devices through intervening private or public networks. Modems, cable modem or Ethernet cards are just a few of the currently available types of network adapters.

1. A biometric monitoring system configured for taking biometric measurements of a patient, comprising:
a biometric measurement unit that is configured to take one or more biometric measurements;
a processor that is coupled to the biometric measurement unit, wherein the processor is operable to receive the biometric measurements from the biometric measurement unit;
a clock that sets timing in the biometric monitoring system; and
a receiver that receives atomic clock time information and the atomic clock time information is used to synchronize the clock.

2. The biometric monitoring system of claim 1, further comprising a storage unit that is coupled to the processor, wherein the storage unit stores the biometric measurements taken by the biometric measurement unit.

3. The biometric monitoring system of claim 1, wherein the processor is further operable to determine when a previous atomic clock time information was received and wherein the receiver is further operable to receive atomic clock time information if the processor determines that the previous atomic clock time information was received outside a predetermined time period.

4. The biometric monitoring system of claim 3, wherein the predetermined time period is seven days.

5. The biometric monitoring system of claim 1, wherein the biometric measurement unit takes the biometric measurements in accordance with a plurality of time intervals.

6. The biometric monitoring system of claim 5, wherein the time intervals include a morning session and an evening session.

7. The biometric monitoring system of claim 1, wherein the biometric measurement is a blood pressure reading, a heart rate, a blood sugar reading, a cholesterol reading or a body temperature reading.

8. The biometric monitoring system of claim 1, further comprising a transceiver that transmits the biometric measurements taken by the biometric measurement unit to a remote location at a defined interval.

9. A biometric monitoring system, comprising:
a receiver that is configured to receive atomic clock time information; and
a processor coupled to the receiver, wherein the processor is operable to time stamp a biometric measurement based on the atomic clock time information.

10. The biometric monitoring system of claim 9, further comprising a biometric measurement unit that is configured to take one or more biometric measurements and forward the biometric measurements to the processor.

11. The biometric monitoring system of claim 10, further comprising a storage unit coupled to the processor, wherein the storage unit is configured to store the biometric measurements for a predetermined time period.

12. The biometric monitoring system of claim 9, further comprising a transceiver configured to at least transmit the stamped biometric measurement to a remote location at a defined interval.

13. In a biometric monitoring system, a method of automatically updating time information, comprising:
receiving atomic clock time information; and
stamping a biometric measurement based on the atomic clock time information.

14. The method of claim 13, wherein receiving the atomic clock time information comprises identifying when a previous atomic clock time information was received and receiving the atomic clock time information if the previous atomic clock time information was received outside a predetermined time period.

15. The method of claim 14, wherein the predetermined time period is seven days.

16. The method of claim 13, further comprising taking the biometric measurement.

17. The method of claim 16, wherein taking the biometric measurement comprises taking a plurality of biometric measurements at multiple intervals and comparing the biometric measurements taken at different intervals with one another.

18. The method of claim 16, further comprising storing the biometric measurements and transmitting the biometric measurements to a remote location at a defined interval.

19. The method of claim 17, wherein the different intervals comprise a morning session and an evening session.

20. The method of claim 13, wherein the biometric measurement is a blood pressure reading, a heart rate, a blood sugar reading, a cholesterol reading or a body temperature reading.
